(19) United States
(12) Patent Application Publication
(10) Pub. No.: US 2025/0101535 A1
(43) Pub. Date: Mar. 27, 2025

(54) SNP MARKER SET FOR DOG IDENTIFICATION AND DOG IDENTIFICATION METHOD USING SAME

(71) Applicant: MEDICLOUD CO., LTD., Seoul (KR)

(72) Inventors: Hyung Ki LEE, Suwon-si (KR); Hwan Jong KWAK, Suwon-si (KR); Byoung Yang KIM, Seoul (KR)

(73) Assignee: MEDICLOUD CO., LTD., Seoul (KR)

(21) Appl. No.: 18/596,884

(22) Filed: Mar. 6, 2024

(30) Foreign Application Priority Data
Sep. 25, 2023 (KR) 10-2023-0128588

(51) Int. Cl. C12Q 1/6888

(52) U.S. Cl. CPC C12Q 1/6888; C12Q 2600/156

(57) ABSTRACT

The present disclosure relates to an SNP marker set for dog identification and a dog identification method using the same. The present disclosure can accurately and easily readably identify individual dogs through the SNP markers on the gene and combinations of the SNPs based on the simple results of AA/AB/BB (Homozygote/Heterozygote/Homo mutant) by using 381 single nucleotide polymorphisms (SNPs) on the gene, thereby proving to be useful for animal individual identification.

### CROSS-REFERENCE TO RELATED APPLICATION

A claim for priority under 35 U.S.C. § 119 is made to Korean Patent Application No. 10-2023-0128588 filed on Sep. 25, 2023 in the Korean Intellectual Property Office, the entire contents of which are hereby incorporated by reference.

### BACKGROUND

### 1. Technical Field

The present disclosure relates to an SNP marker set for dog identification and a dog identification method using the same, and more specifically, to an SNP marker set for dog identification with improved accuracy and readability, and a dog identification method using the same.

### 2. Description of Related Art

Rapid growth in industry and economy has caused social and/or psychological problems. In order to alleviate stress and anxiety, the number of households keeping pets has increased, so it has led to the rapid development of the pet industry, including pet insurance. However, the growth of the pet industry has led to social, ethical, and industrial problems.

Among the problems, the loss and identification of pets have become major issues. When pets are lost accidentally, regardless of owners' intentions, there is a limitation in identifying the pet's identity through appearance. Moreover, wandering pets cause social problems due to their aggression, excrements, and rummage through garbage. Furthermore, in the United States, about 6.5 million pets including outdoor animals are gathered in animal shelters, and 1.5 million pets are euthanized annually. In England, pet insurance fraud increased fourfold in 2011, and has become a social problem.

The root cause of the problems is the lack of official records of pet ownership. In China, the government is reviewing what kind of identity information about pets should be databased. One of currently developed methods is subcutaneous microchip injection, which is a method of identifying pets through serial numbers embedded in the chip.

However, since chip readers are easy to buy and use, the chip readers may be a target of crime, and the information about the pet owner, stored on the chip, may be easily used for fraud. Additionally, transplanting microchips into other pets can be another route for pet insurance fraud, and if the loss of a pet is intentional, the chip can be removed by the owner, so there may be a limitation in assigning responsibility for social problems caused by the pet.

Another method for identifying pets and minimizing the limitations of microchips is based on genetic markers. The method uses the pet's genetic information, so the pet can be identified and its owner found if there are records of the pet. For the method based on genetic markers, various genetic markers have been used to prevent genetic polymorphism, heterozygosity, and phylogeny. The markers can include autosomal microsatellite markers, Y chromosome markers, mitochondrial DNA (mtDNA), and single nucleotide polymorphisms (SNPs).

Among the markers, SNPs are abundant in the genome and have a lower mutation rate compared to the polymorphic microsatellites and the mtDNA, and once discovered, can be analyzed efficiently. SNPs not only occur frequently in mammalian genomes and are useful for rapid, large-scale, and economical genotyping, but also can be effectively used as tools for ecological, conservation, population, and evolutionary studies.

As of 2018, the number of domestic dogs in Korea is estimated to be approximately 6.35 million, and in the top 20 countries worldwide for pet dogs, there are an estimated total of 578.83 million individuals. The most commonly used method for dog identification is the use of short tandem repeat markers (STR Markers), which are highly accurate but have the problem that a plurality of genotypes can be identified from a single marker, and variations due to humidity and temperature can affect genotype identification.

With the above background, the inventors of the present disclosure have conducted research to accurately distinguish and identify specific dogs among pets from others using 381 single nucleotide polymorphism (SNP) markers on the gene without using STR markers. The research showed that individual dogs could be accurately and easily readably identified through the SNP markers on the gene and combinations of the SNPs based on the simple results of AA/AB/BB (Homozygote/Heterozygote/Homo mutant).

### SUMMARY

The present disclosure has been made to solve the above-mentioned problems occurring in the prior art, and in an aspect of the present disclosure, an objective of the present disclosure is to provide an SNP marker set for dog identification including one or more SNPs selected from a group consisting of 381 SNPs from a dog genome dataset.

Another objective of the present disclosure is to provide a composition for dog identification including an agent capable of detecting or amplifying the SNP marker set for dog identification.

Another objective of the present disclosure is to provide a kit or a microarray for dog identification including the composition for dog identification.

Another objective of the present disclosure is to provide a dog identification method including: operation i) extracting nucleic acids from a dog intended for individual identification; and operation ii) confirming the SNP marker set in the extracted nucleic acids.

The advantages and features of the present disclosure and methods accomplishing the advantages and features will become apparent from the following detailed description of exemplary embodiments with reference to the accompanying drawings. However, the present disclosure is not limited to exemplary embodiment disclosed herein but will be implemented in various forms. The exemplary embodiments are provided so that the present disclosure is completely disclosed, and a person of ordinary skilled in the art will fully understand the scope of the present disclosure. Therefore, the present disclosure will be defined only by the scope of the appended claims.

Terms used in the specification are used to describe specific embodiments of the present disclosure and are not intended to limit the scope of the present disclosure. In the specification, the terms of a singular form may include plural forms unless otherwise specified. It should be also understood that the terms of ‘include’ or ‘have’ in the specification are used to mean that there is no intent to exclude existence or addition of other components besides components described in the specification. In the detailed description, the same reference numbers of the drawings refer to the same or equivalent parts of the present disclosure, and the term “and/or” is understood to include a combination of one or more of components described above. It will be understood that terms, such as “first” or “second” may be used in the specification to describe various components but are not restricted to the above terms. The terms may be used to discriminate one component from another component. Therefore, of course, the first component may be named as the second component within the scope of the present disclosure.

Unless otherwise defined, all terms (including technical and scientific terms) used herein have the same meaning as commonly understood by those skilled in the technical field to which the present disclosure pertains. It will be further understood that terms, such as those defined in commonly used dictionaries, should not be interpreted in an idealized or overly formal sense unless expressly so defined herein.

The present disclosure provides an SNP marker set for dog identification, which includes all 381 SNPs listed in Table 41 in a dog genome dataset.

SNP of the present disclosure refers to single nucleotide polymorphism, and means the diversity in DNA sequences that occur when a single nucleotide (A, T, C, or G) in the genome differs among individuals of the same species or between the homologous chromosomes of an individual.

For instance, in a case in which there are differences in single nucleotides like three DNA fragments (e.g., AAGT[A/A]AG, AAGT[A/G]AG, AAGT[G/G]AG) of different individuals, they are referred to as two alleles (A or G), and most SNPs typically have two alleles.

Within one population, SNPs can be assigned a minor allele frequency (MAF), which is the lowest allele frequency at a locus found in a specific population. The single nucleotide can be changed (substituted), deleted, or added (inserted) in the polynucleotide sequence, and the SNPs can cause changes in the translation reading frame. In this specification, the term “SNP marker” may be interchangeably used with “SNP”.

The marker in the present disclosure refers to a biomarker, and specifically, to a marker for identifying individual dogs in the present disclosure.

As the number of SNP markers included in the marker set of the present disclosure increases, the number of possibilities for individual identification increases, thereby improving accuracy.

Each SNP marker constituting the SNP marker set is the SNP marker listed in the above table, and those skilled in the art can easily understand the position on the dog genome and the surrounding sequence based on the information listed.

In a specific embodiment of the present disclosure, it has been confirmed that the SNP marker set of the present disclosure can identify a specific individual with 100% accuracy.

The nucleotide sequences used in the present disclosure are interpreted as including sequences that exhibit substantial identity to the listed sequences, considering biologically equivalent variants. The substantial identity means a sequence that, when the sequence of the present disclosure and another sequence, different from the above sequence, are aligned to correspond to each other and the aligned sequence is analyzed using algorithms commonly employed in the art, exhibits at least 60% homology, specifically 70% homology, more specifically 80% homology, and even more specifically 90% homology.

The alleles of the present disclosure are nucleotide sequences located in a pair at a locus on a pair of homologous chromosomes, and different alleles can sometimes exhibit different traits.

The nucleotides of the present disclosure are deoxyribonucleotides or ribonucleotides existing in a single-stranded or double-stranded form, and can include natural nucleotide analogs.

The breed of the dog is not limited, but can be preferably selected from a group including Beagle, Water dog, Maltese, Pomeranian, Poodle, Yorkshire Terrier, Shih Tzu, Dachshund, Chihuahua, American Pitbull Terrier, and crossbreeds thereof, and more preferably, can be Beagle, Chihuahua, or American Pitbull Terrier.

Moreover, the present disclosure provides a composition for dog identification, which includes an agent capable of detecting or amplifying the SNP marker set for dog identification.

The amplification in the present disclosure refers to the process that, to activate expression of a specific trait in a cell, a portion of a chromosome containing genes or a group of genes governing the specific trait increases the number of the chromosomes by repeating specific replication.

The agent capable of detecting the SNP markers of the present disclosure means an agent that can specifically bind to and recognize the SNP markers included in the SNP marker composition, or an agent that can detect and amplify the SNPs.

The agent can be a probe that can specifically bind to the polymorphic site containing the SNP markers, or a primer or a primer set that can specifically detect and amplify the polynucleotide containing the SNP markers or complementary polynucleotide.

The probe of the present disclosure refers to a natural or modified monomer or a linear oligomer of linkages, includes deoxyribonucleotides and ribonucleotides, can specifically hybridize with the target nucleotide sequence, and can naturally occur or can be artificially synthesized. The probe according to the present disclosure can be single-stranded, preferably can be an oligodeoxyribonucleotide. The probe can include natural dNMPs (i.e., dAMP, dGMP, dCMP, and dTMP), nucleotide analogs, or derivatives. Additionally, the probe may further include ribonucleotides.

The primer of the present disclosure refers to a single-stranded oligonucleotide which can act as a starting point for template-directed DNA synthesis under suitable conditions (i.e., four different nucleoside triphosphates and polymerase reaction enzymes) at an appropriate temperature and in an appropriate buffer solution. The appropriate length of the primer can vary according to various elements, for instance, temperature and uses of the primer. Moreover, the sequence of the primer does not need to be entirely complementary to a portion of the template sequence, but is enough to have sufficient complementarity within a range sufficient to hybridize with the template and perform the unique function of the primer. Therefore, the primer in the present disclosure does not need to be perfectly complementary to the nucleotide sequence of the template gene, but is enough to have sufficient complementarity to hybridize with the gene sequence and function as a primer. Furthermore, the primer according to the present disclosure is preferably usable in a gene amplification reaction. The amplification reaction refers to a reaction which amplifies nucleic acid molecules, and the gene amplification reaction is well known in the art, and may include, for example, polymerase chain reaction (PCR), reverse transcriptase PCR (RT-PCR), ligase chain reaction (LCR), transcription mediated amplification (TMA), nucleic acid sequence-based amplification (NASBA), etc.

In this instance, the sequence of the primer can be modified based on the knowledge in the art regarding PCR conditions, and the lengths of the sense and antisense primers.

As a specific example, there are methylation, capping, substitution of nucleotides, or modification between nucleotides, for instance, modification into uncharged linkages (e.g., methyl phosphonate, phosphotriester, phosphoramidate, and carbamate) or charged linkages (e.g., phosphorothioate, and phosphorodithioate).

The sequence of the primer can be modified to enhance specificity and binding force to the SNP marker, and a plurality of primers with different nucleotide sequences for the same SNP marker can be used. More specifically, the length of each primer can be adjusted by adding repeating nucleotides to the distal end of the primer, and the type of nucleotide can be A, T, G, C, or a combination thereof, but there is no restriction in the type of nucleotide as long as the sequence is repeating.

In addition, the present disclosure provides a kit for dog identification, which includes the composition for dog identification.

The kit of the present disclosure can distinguish and identify individual dogs by detecting and confirming the SNP markers of the present disclosure.

The kit may be a PCR kit containing essential elements required to perform PCR. For example, the PCR kit can include, in addition to the specific primers for the SNP markers, test tubes or other appropriate containers, a reaction buffer solution (with various pHs and magnesium concentrations), deoxynucleotides (dNTPs), dideoxynucleotides (ddNTPs), enzymes such as Taq polymerase and reverse transcriptase enzymes, DNase, RNAse inhibitors, DEPC-water, sterile water, etc. Furthermore, the PCR kit may also include a pair of gene-specific primers used as a quantitative control group.

Moreover, the present disclosure provides a microarray for dog identification, which includes the composition for dog identification.

The microarray of the present disclosure may include DNA or RNA polynucleotides. The microarray is made up of typical microarrays except for including the polynucleotides of the present disclosure in the probe polynucleotides. A method for manufacturing a microarray by immobilizing probe polynucleotides on a substrate is well known in the art.

Hybridization of nucleic acids on the microarray and detection of the hybridization results are well known in the art. The hybridization results can be detected, for example, by labeling a nucleic acid sample as a marker material which includes a fluorescent material and can generate a detectable signal, hybridizing the sample on the microarray, and detecting the signal generated from the marker material. The fluorescent material may include pyrene, Texas red, fluorescein, BODYPY, tetramethylrhodamine, alexa, cyanine, allophycocyanin, nile blue, nile red, or other fluorescent materials that emit fluorescence. Additionally, a fluorescent material with a high quantum yield can be used. In addition, the fluorescent material can be hydrophilic or hydrophobic dye.

Moreover, the present disclosure provides a dog identification method, which includes: operation i) of extracting nucleic acids from a dog intended for individual identification; and operation ii) of confirming the SNP marker set in the extracted nucleic acids.

The nucleic acids of the present disclosure can be extracted from a sample obtained from the dog to be identified, and can be extracted from a sample selected from a group consisting of the blood, epidermis, urine, feces, hair, saliva, oral cells, muscles, and organs of the dog.

The extraction of genomic DNA from the sample can be carried out using commonly used methods in the art, such as phenol/chloroform extraction, SDS extraction, CTAB extraction, or commercially available DNA extraction kits.

The extracted nucleic acids can be used as templates, and a target sequence can be amplified by performing an amplification reaction using a specific primer set. The method for amplifying target nucleic acids may be a method for amplifying through the polymerase chain reaction (PCR), the ligase chain reaction (LCR), the nucleic acid sequence-based amplification (NASBA), the transcription-based amplification system, the strand displacement amplification, or the amplification through QP replicase, or any other method known in the art for amplifying nucleic acid molecules. Thereamong, PCR is a method that amplifies the target nucleic acid from the primer set that specifically bind to the target nucleic acid using polymerases. The PCR method is well known in the art, and can also utilize commercially available kits. The PCR can be performed using a PCR reaction mixture containing various components known in the art that are necessary for the PCR reaction.

Operation ii) of the present disclosure can be realized by applying various methods known in the art for elucidating specific sequences, namely identifying the bases of the SNP. For instance, the methods can include a fluorescent in situ hybridization (FISH), a DNA sequencing, a PFGE analysis, a Southern blot analysis, a single-strand conformation analysis (SSCA), an RNase protection analysis, a dot blot analysis, a denaturing gradient gel electrophoresis (DGGE), a method using protein (e.g., mutS protein of E. coli) that recognizes a nucleotide mismatch, an allele-specific probe hybridization, an allele-specific amplification, a 5′ nuclease digestion, a molecular beacon assay, an oligonucleotide ligation assays, a size analysis, a single-stranded conformation polymorphism (SSCP), an allele-specific PCR, and an analysis by mass array, but are not limited thereto.

After operation ii), the method may further include operation of verifying one or more pieces of information selected from a group consisting of the blood type, physical characteristics, weight, sex, and age of the dog.

The results of the dog identification of the present disclosure can be transferred to a client by typical format and method known in the art, for example, through means such as telephone, letter, e-mail, or web page publication, but are not limited thereto.

The test results may provide quantification of the degree of identification or may only provide the identification results. The test results may include not only information about the dog but also information about a dog owner.

### DETAILED DESCRIPTION

Hereinafter, the contents of the present disclosure will be described in more detail through the following embodiments and experimental examples. However, the scope of the present disclosure is not limited to the following examples and experimental examples, and includes modifications equivalent to such technical ideas.

### Embodiment 1. Sample Preparation and DNA Extraction

Specimens used in the present disclosure were six dogs in a family relationship. DNA extraction was performed from blood samples of the six dogs by using an Exgene Clinic SV kit (GeneAll). The six dogs were two Chihuahuas, three Beagles, and one American Pitbull Terrier.

### Embodiment 2. SNP Genotyping

ProFlex PCR system (applied biosystems) multiplex PCR was performed to the DNA extracted in Embodiment 1 by using the i-StarMAX™ II DNA Polymerase kit (iNtRON).

For Library construction used in NGS, using 1 ng of DNA and an ampliseq library kit, a reaction was carried out under the conditions of starting at 95° C. for 5 minutes and performing 30 cycles of 95° C. for 30 seconds, 60° C. for 75 seconds, and 72° C. for 30 seconds, and then, a final reaction was carried out at 72° C. for 5 minutes.

### Experimental Example 1. Dog Identification Using Selected SNP Markers

The product amplified through the PCR in Embodiment 2 was experimented on in an IonS5 sequencing machine to acquire the genotype of each sample, and then, individual dogs were identified based on the genotype.

### 1-1. Preliminary Test on Dog Population

Using 381 single nucleotide polymorphisms (SNPs) on the gene, it was confirmed that accuracy due to easiness in reading the results was enhanced, based on the simple results of AA/AB/BB (Homozygote/Heterozygote/Homo mutant).

Additionally, a genotyping assay using TagMan probes was used to increase the ease of reading via Next Generation Sequencing (NGS) for SNPs that may not have all three (AA/AB/BB) genotypes and may be difficult to read.

Therefore, differences among individuals as shown in Table 1 below could be confirmed.

TABLE 1

| | | | Beagle | Beagle | Beagle | |
|---|---|---|---|---|---|---|
| 381 Markers | Gyeomdung | Lotto | Mom | Dad | Daughter | Mungchi |
| Gyeomdung | 1.000 | 0.749 | 0.630 | 0.618 | 0.573 | 0.608 |
| (1) | | | | | | |
| Lotto | 0.749 | 1.000 | 0.643 | 0.614 | 0.604 | 0.604 |
| (1-1) | | | | | | |
| Beagle Mom | 0.630 | 0.643 | 1.000 | 0.798 | 0.807 | 0.630 |
| (2) | | | | | | |
| Beagle Dad | 0.618 | 0.614 | 0.798 | 1.000 | 0.811 | 0.627 |
| (3) | | | | | | |
| Beagle | 0.573 | 0.604 | 0.807 | 0.811 | 1.000 | 0.591 |
| Daughter | | | | | | |
| (2-1) | | | | | | |
| Mungchi | 0.608 | 0.604 | 0.630 | 0.627 | 0.591 | 1.000 |
| (4) | | | | | | |

### 1-2. SNP Selection for Each Individual

381 SNPs on the gene were selected, and as shown in Tables 2 to 40 below, the genotype was acquired from each sample, and it was confirmed that individuals could be identified based on the genotype.

TABLE 2
Chromosome 1

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 1 | CHR1 | 3962719 | A | G | A/A | A/G | G/G | G/G | G/G | G/G |
| 2 | CHR1 | 12076349 | C | T | T/T | T/T | C/T | C/T | C/T | C/T |
| 3 | CHR1 | 20842130 | C | T | C/T | C/T | C/C | C/C | C/C | T/T |
| 4 | CHR1 | 44484310 | T | C | T/T | T/T | T/T | T/T | T/T | C/C |
| 5 | CHR1 | 54192143 | G | C | G/G | G/G | G/G | G/G | G/G | G/G |
| 6 | CHR1 | 69171090 | A | G | A/G | A/A | A/G | A/G | A/G | A/G |
| 7 | CHR1 | 70238933 | C | T | C/T | C/T | C/C | C/C | C/C | T/T |
| 8 | CHR1 | 72613047 | T | C | T/C | T/C | T/C | T/C | T/C | T/T |
| 9 | CHR1 | 74450772 | G | A | A/A | G/A | A/A | A/A | A/A | G/A |
| 10 | CHR1 | 80971770 | A | G | A/A | A/A | A/A | A/A | A/A | A/G |
| 11 | CHR1 | 105514131 | G | C | G/C | G/C | G/C | G/G | G/G | G/G |
| 12 | CHR1 | 106430955 | G | A | A/A | G/A | G/A | A/A | A/A | G/A |
| 13 | CHR1 | 117495849 | C | A | A/A | C/A | C/C | C/A | C/C | C/A |
| 14 | CHR1 | 119306331 | A | G | G/G | G/G | A/G | G/G | G/G | G/G |
| 15 | CHR1 | 119414584 | G | A | A/A | G/A | G/A | G/G | G/G | G/A |

TABLE 3
Chromosome 2

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 16 | CHR2 | 2610859 | T | C | T/C | T/C | T/C | T/C | T/C | T/T |
| 17 | CHR2 | 38293797 | A | G | A/G | A/G | G/G | G/G | G/G | A/G |
| 18 | CHR2 | 67888039 | A | G | A/A | A/G | A/A | A/A | A/A | A/G |
| 19 | CHR2 | 77806065 | G | A | G/A | A/A | G/A | G/A | G/A | A/A |
| 20 | CHR2 | 84535676 | G | A | G/G | G/A | G/G | G/G | G/G | G/G |

TABLE 4
Chromosome 3

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 21 | CHR3 | 1252765 | C | A | C/A | C/A | C/A | A/A | A/A | A/A |
| 22 | CHR3 | 10255068 | C | T | C/T | T/T | C/C | C/T | C/C | C/C |
| 23 | CHR3 | 24757939 | G | A | G/A | G/A | G/A | A/A | G/A | A/A |
| 24 | CHR3 | 28054239 | G | A | G/A | G/A | G/A | G/G | G/G | A/A |
| 25 | CHR3 | 37849557 | C | T | C/C | C/T | T/T | C/T | T/T | T/T |
| 26 | CHR3 | 40302288 | A | G | A/G | A/G | G/G | A/A | A/G | G/G |
| 27 | CHR3 | 41849479 | G | A | G/G | G/G | G/A | G/G | G/G | G/G |
| 28 | CHR3 | 43055696 | A | G | A/G | A/G | A/G | A/G | A/A | A/A |
| 29 | CHR3 | 43063677 | T | C | T/C | T/C | T/C | T/C | T/T | T/T |
| 30 | CHR3 | 44126666 | G | C | C/C | C/C | C/C | G/C | G/C | G/C |
| 31 | CHR3 | 60621051 | T | C | T/T | T/T | T/C | T/C | T/C | C/C |
| 32 | CHR3 | 64084413 | G | A | G/A | A/A | G/A | G/A | G/A | G/G |
| 33 | CHR3 | 73570828 | T | C | T/C | T/T | T/C | T/C | T/C | T/C |
| 34 | CHR3 | 90291255 | T | C | C/C | C/C | T/T | T/T | T/T | T/T |
| 35 | CHR3 | 91103945 | G | T | G/G | G/G | G/G | G/G | G/G | G/G |
| 36 | CHR3 | 91626907 | C | T | C/T | C/C | T/T | T/T | T/T | T/T |

TABLE 5
Chromosome 4

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 37 | CHR4 | 10582893 | A | T | A/T | A/A | T/T | T/T | T/T | T/T |
| 38 | CHR4 | 31301072 | G | A | A/A | A/A | G/G | G/G | G/G | G/A |
| 39 | CHR4 | 39182836 | A | T | T/T | A/T | A/T | A/T | T/T | T/T |
| 40 | CHR4 | 42104780 | T | C | T/T | T/T | T/T | T/C | T/T | T/T |
| 41 | CHR4 | 53911120 | G | C | C/C | G/C | G/C | G/C | G/G | G/C |
| 42 | CHR4 | 64121754 | T | C | T/C | T/T | T/C | T/C | T/T | T/C |
| 43 | CHR4 | 67040898 | C | T | C/T | C/T | T/T | T/T | T/T | C/C |
| 44 | CHR4 | 67040939 | G | A | G/G | G/G | G/G | G/A | G/G | G/G |
| 45 | CHR4 | 70217695 | G | A | G/A | G/G | G/G | G/A | G/G | A/A |
| 46 | CHR4 | 75910211 | A | G | G/G | G/G | A/G | A/G | G/G | G/G |
| 47 | CHR4 | 86049027 | C | T | T/T | C/T | T/T | C/T | T/T | T/T |
| 48 | CHR4 | 86998873 | C | T | C/C | C/C | C/T | C/C | C/C | T/T |

TABLE 6
Chromosome 5

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 49 | CHR5 | 5410890 | C | T | C/T | C/T | T/T | T/T | T/T | T/T |
| 50 | CHR5 | 13080303 | A | G | A/G | A/G | A/G | A/A | A/G | A/G |
| 51 | CHR5 | 25193842 | G | A | A/A | A/A | G/A | G/A | A/A | A/A |
| 52 | CHR5 | 26320165 | C | T | T/T | C/T | C/T | T/T | T/T | T/T |
| 53 | CHR5 | 36642434 | A | G | A/A | A/A | A/A | A/G | A/G | A/G |
| 54 | CHR5 | 44642087 | A | T | A/A | A/T | A/T | T/T | T/T | A/T |
| 55 | CHR5 | 44650576 | G | A | A/A | G/A | G/A | G/G | G/G | G/A |
| 56 | CHR5 | 50794117 | G | C | G/G | G/C | G/G | G/C | S/C | G/C |
| 57 | CHR5 | 55349573 | A | G | A/G | A/G | A/G | A/A | A/A | A/G |
| 58 | CHR5 | 56989849 | A | G | A/A | A/G | A/G | G/G | A/G | G/G |
| 59 | CHR5 | 63694334 | G | A | G/G | G/A | G/G | G/G | G/G | G/G |
| 60 | CHR5 | 63694460 | C | T | T/T | T/T | T/T | T/T | T/T | C/C |
| 61 | CHR5 | 64611038 | G | A | G/A | A/A | G/G | G/G | Nocall | G/A |
| 62 | CHR5 | 79189887 | C | A | C/A | C/A | C/A | C/A | C/C | A/A |
| 63 | CHR5 | 85451804 | A | G | G/G | A/G | A/A | A/A | A/A | A/A |
| 64 | CHR5 | 87925312 | A | G | A/G | A/G | A/A | A/A | A/A | A/A |
| 65 | CHR5 | 88910280 | T | C | T/T | T/C | T/C | T/C | C/C | T/T |

TABLE 7
Chromosome 6

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 66 | CHR6 | 11553458 | A | G | A/G | A/G | A/G | A/G | A/G | A/G |
| 67 | CHR6 | 16653546 | G | C | G/C | G/G | G/C | G/C | G/C | G/G |
| 68 | CHR6 | 33976751 | T | C | T/T | T/T | T/C | T/C | T/T | T/C |
| 69 | CHR6 | 44672748 | A | G | A/G | A/G | A/A | A/A | A/A | A/A |
| 70 | CHR6 | 64006720 | A | G | G/G | A/G | A/A | G/G | A/G | A/A |
| 71 | CHR6 | 64403024 | G | A | G/G | G/A | G/A | G/G | G/G | G/A |
| 72 | CHR6 | 74727390 | G | C | G/C | G/G | C/C | C/C | C/C | G/G |

TABLE 8
Chromosome 7

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 73 | CHR7 | 76294 | A | G | A/G | A/G | A/A | A/A | A/A | A/A |
| 74 | CHR7 | 3318809 | A | G | A/A | A/G | G/G | G/G | G/G | G/G |
| 75 | CHR7 | 6423299 | C | T | C/T | C/T | C/T | C/T | C/T | C/C |
| 76 | CHR7 | 15011628 | A | G | G/G | G/G | G/G | A/G | A/G | A/A |
| 77 | CHR7 | 15017979 | G | A | G/G | G/G | G/G | G/A | G/A | A/A |
| 78 | CHR7 | 36171712 | T | C | T/C | T/T | C/C | C/C | C/C | C/C |
| 79 | CHR7 | 36555518 | T | C | T/T | T/C | T/T | T/C | T/C | T/C |
| 80 | CHR7 | 38350972 | C | G | G/G | C/G | C/G | C/G | C/C | C/C |
| 81 | CHR7 | 46413871 | C | T | C/T | C/T | C/C | C/C | C/C | T/T |
| 82 | CHR7 | 63919631 | G | C | G/G | Nocall | C/C | G/C | G/C | G/G |
| 83 | CHR7 | 76487265 | G | A | A/A | A/A | G/A | G/A | G/G | A/A |
| 84 | CHR7 | 80017081 | G | A | A/A | A/A | A/A | A/A | A/A | G/G |
| 85 | CHR7 | 80443374 | A | G | A/G | A/G | A/G | A/G | G/G | A/A |

TABLE 9
Chromosome 8

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 86 | CHR8 | 5291824 | A | G | A/G | A/G | A/A | A/A | A/A | A/G |
| 87 | CHR8 | 6188937 | G | A | G/A | G/A | G/A | G/A | G/G | G/A |
| 88 | CHR8 | 6267365 | G | T | G/T | Nocall | G/T | G/G | G/G | G/T |
| 89 | CHR8 | 10044405 | C | T | T/T | C/T | T/T | T/T | T/T | T/T |
| 90 | CHR8 | 10413839 | G | A | A/A | G/A | G/G | G/A | G/G | A/A |
| 91 | CHR8 | 18121580 | C | T | T/T | T/T | C/C | C/C | C/C | C/C |
| 92 | CHR8 | 19076567 | T | C | T/C | T/C | T/T | T/C | T/T | T/T |
| 93 | CHR8 | 24614720 | G | A | G/G | G/A | G/G | G/G | G/G | A/A |
| 94 | CHR8 | 41635950 | T | A | T/A | T/A | A/A | T/A | A/A | T/T |
| 95 | CHR8 | 45852939 | A | G | A/A | A/A | A/A | A/A | A/A | G/G |
| 96 | CHR8 | 52381322 | G | A | G/A | G/A | G/A | G/A | A/A | G/G |
| 97 | CHR8 | 63196958 | G | A | G/A | G/G | G/A | A/A | A/A | G/G |
| 98 | CHR8 | 67183794 | C | T | C/T | T/T | T/T | C/T | T/T | C/T |
| 99 | CHR8 | 71834501 | G | C | G/G | G/G | G/G | G/G | G/G | G/G |

TABLE 10
Chromosome 9

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 100 | CHR9 | 10346478 | G | A | G/A | G/A | G/A | G/A | G/A | G/A |
| 101 | CHR9 | 20867959 | C | T | T/T | T/T | C/T | T/T | T/T | C/T |
| 102 | CHR9 | 22610227 | A | G | A/G | A/A | A/G | A/G | G/G | A/G |
| 103 | CHR9 | 32506288 | G | A | G/A | G/G | G/A | G/A | G/A | G/A |
| 104 | CHR9 | 40096141 | T | C | T/T | T/T | T/C | T/C | T/C | T/T |
| 105 | CHR9 | 40702757 | T | C | C/C | C/C | T/C | T/C | T/C | C/C |
| 106 | CHR9 | 50114927 | G | A | A/A | G/A | G/A | G/A | G/A | A/A |
| 107 | CHR9 | 52710991 | C | T | T/T | C/T | C/C | C/C | C/C | C/C |
| 108 | CHR9 | 56021221 | A | G | A/G | A/G | A/G | A/G | A/A | A/G |
| 109 | CHR9 | 60437147 | A | G | A/G | G/G | A/G | A/G | G/G | A/A |

TABLE 11
Chromosome 10

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 110 | CHR10 | 8085469 | C | T | T/T | T/T | C/C | C/C | C/C | T/T |
| 111 | CHR10 | 8183593 | C | T | C/T | T/T | C/T | C/T | C/T | C/C |
| 112 | CHR10 | 10652659 | C | T | T/T | C/T | C/T | C/T | C/T | C/T |
| 113 | CHR10 | 14685262 | C | T | T/T | T/T | T/T | T/T | T/T | C/C |
| 114 | CHR10 | 15113284 | T | A | T/T | T/T | T/A | T/A | T/A | T/A |
| 115 | CHR10 | 15362456 | A | G | G/G | G/G | A/G | A/G | A/G | A/G |
| 116 | CHR10 | 22409408 | A | G | A/A | A/G | A/A | A/G | A/A | A/G |
| 117 | CHR10 | 30034450 | A | G | A/G | G/G | G/G | A/G | G/G | G/G |
| 118 | CHR10 | 35942926 | T | C | T/C | T/C | T/C | T/C | T/C | T/C |
| 119 | CHR10 | 39548483 | C | T | T/T | T/T | C/C | C/C | C/C | C/C |
| 120 | CHR10 | 47923623 | C | T | C/C | C/T | C/T | C/T | C/T | C/T |
| 121 | CHR10 | 49188334 | C | T | C/C | C/C | C/T | C/T | C/T | C/C |
| 122 | CHR10 | 57954366 | C | T | T/T | C/C | C/C | C/C | C/C | T/T |
| 123 | CHR10 | 66922269 | T | C | T/T | T/C | T/C | T/C | T/T | T/C |
| 124 | CHR10 | 67126524 | G | A | A/A | A/A | G/A | G/A | A/A | G/A |
| 125 | CHR10 | 68918209 | C | T | C/C | C/T | C/C | C/C | C/C | T/T |

TABLE 12
Chromosome 11

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 126 | CHR11 | 1161870 | G | A | G/A | A/A | G/A | G/A | G/G | A/A |
| 127 | CHR11 | 3242754 | A | C | A/C | C/C | C/C | C/C | C/C | C/C |
| 128 | CHR11 | 5318488 | C | T | C/C | C/T | T/T | T/T | T/T | C/T |
| 129 | CHR11 | 12479869 | C | T | C/C | C/C | C/C | C/C | C/C | C/T |
| 130 | CHR11 | 23907101 | A | C | A/A | A/A | A/A | A/C | A/C | A/C |
| 131 | CHR11 | 33317810 | T | A | T/T | T/T | T/T | T/T | T/T | T/T |
| 132 | CHR11 | 33326685 | C | T | C/T | C/C | C/C | C/C | C/C | C/T |
| 133 | CHR11 | 33326726 | CCT | — | CCT/ | CCT/ | CCT/ | CCT/ | CCT/ | CCT/ |
| | | | | | CCT | CCT | CCT | CCT | CCT | CCT |
| 134 | CHR11 | 62157625 | G | A | G/A | A/A | G/A | G/A | G/G | A/A |
| 135 | CHR11 | 65603333 | C | A | C/A | C/A | A/A | A/A | A/A | A/A |
| 136 | CHR11 | 70698603 | G | T | G/G | G/G | G/T | T/T | G/T | T/T |
| 137 | CHR11 | 72333665 | G | C | G/C | C/C | G/C | G/C | G/C | C/C |
| 138 | CHR11 | 73866275 | C | G | C/C | C/G | C/G | G/G | G/G | C/C |

TABLE 13
Chromosome 12

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 139 | CHR12 | 186569 | C | G | C/C | C/G | C/C | C/C | C/C | C/C |
| 140 | CHR12 | 5579055 | A | G | G/G | A/G | A/A | A/A | A/A | G/G |
| 141 | CHR12 | 8532712 | A | G | A/G | A/G | A/G | A/G | A/G | A/A |
| 142 | CHR12 | 10683432 | T | G | G/G | G/G | T/G | G/G | T/G | G/G |
| 143 | CHR12 | 23059939 | A | G | G/G | G/G | A/G | A/G | A/G | G/G |
| 144 | CHR12 | 35306641 | A | G | A/A | A/G | G/G | A/G | G/G | A/A |
| 145 | CHR12 | 40681020 | G | A | G/G | G/A | G/A | A/A | A/A | G/G |
| 146 | CHR12 | 55201839 | G | A | G/A | A/A | G/A | G/A | A/A | G/A |
| 147 | CHR12 | 68125319 | A | G | A/A | A/A | A/G | A/G | G/G | A/A |
| 148 | CHR12 | 70657733 | G | A | G/A | A/A | G/A | G/A | G/G | G/A |
| 149 | CHR12 | 72440787 | A | G | G/G | G/G | G/G | G/G | G/G | G/G |

TABLE 14
Chromosome 13

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 150 | CHR13 | 2561486 | C | T | C/C | C/C | C/C | C/C | C/C | C/C |
| 151 | CHR13 | 7791160 | T | A | T/A | A/A | T/T | T/T | T/T | T/A |
| 152 | CHR13 | 8704192 | T | C | C/C | T/C | T/T | T/T | T/T | T/T |
| 153 | CHR13 | 40584681 | G | C | G/C | G/C | G/G | G/G | G/G | G/C |
| 154 | CHR13 | 40616856 | T | C | T/C | T/C | T/C | T/C | C/C | C/C |
| 155 | CHR13 | 44869343 | T | G | G/G | T/G | T/T | T/T | T/T | T/G |
| 156 | CHR13 | 59896033 | T | G | T/T | T/T | T/G | T/G | T/T | T/G |
| 157 | CHR13 | 62898269 | G | A | G/G | G/G | G/G | G/G | G/G | G/G |

TABLE 15
Chromosome 14

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 158 | CHR14 | 7319133 | C | A | C/C | C/C | C/C | C/C | C/C | C/A |
| 159 | CHR14 | 13726593 | ATCT | — | ATCT/ | ATCT/ | ATCT/ | ATCT/ | ATCT/ | ATCT/ |
| | | | | | ATCT | ATCT | ATCT | ATCT | ATCT | ATCT |
| 160 | CHR14 | 21072223 | C | T | C/C | C/C | C/C | C/T | C/C | C/T |
| 161 | CHR14 | 25812525 | C | T | C/T | C/T | C/C | C/T | C/C | C/C |
| 162 | CHR14 | 43370655 | A | G | G/G | A/G | A/A | A/A | A/A | A/G |
| 163 | CHR14 | 50063321 | A | G | A/G | A/A | A/G | A/G | A/A | G/G |
| 164 | CHR14 | 55735620 | C | T | C/C | C/T | T/T | T/T | T/T | C/C |
| 165 | CHR14 | 58465266 | A | G | A/G | Nocall | A/G | A/G | G/G | G/G |

TABLE 16
Chromosome 15

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 166 | CHR15 | 1165219 | C | T | C/C | C/C | C/C | C/C | C/C | C/C |
| 167 | CHR15 | 19299365 | T | C | C/C | T/C | C/C | C/C | C/C | T/T |
| 168 | CHR15 | 22834903 | C | A | C/A | C/A | C/A | C/A | C/A | C/C |
| 169 | CHR15 | 31936820 | T | A | T/A | T/A | A/A | A/A | A/A | T/T |
| 170 | CHR15 | 61796402 | G | T | G/G | G/G | G/G | G/G | G/G | G/T |

TABLE 17
Chromosome 16

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 171 | CHR16 | 29634940 | T | C | C/C | T/C | T/C | T/C | T/T | T/C |
| 172 | CHR16 | 29675662 | C | A | C/A | C/A | C/C | C/C | C/C | C/A |
| 173 | CHR16 | 38237959 | A | T | T/T | A/T | T/T | A/T | T/T | A/A |
| 174 | CHR16 | 46884446 | T | G | T/G | T/G | T/T | T/G | T/T | T/T |
| 175 | CHR16 | 57958947 | C | T | C/T | C/C | T/T | C/T | C/T | T/T |
| 176 | CHR16 | 58093031 | T | G | T/G | G/G | T/G | T/G | T/G | T/G |

TABLE 18
Chromosome 17

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 177 | CHR17 | 7140289 | G | C | G/C | C/C | G/G | G/C | G/G | G/C |
| 178 | CHR17 | 9407683 | G | A | A/A | A/A | G/A | G/G | G/G | G/A |
| 179 | CHR17 | 10050848 | T | C | C/C | C/C | T/C | T/T | T/C | T/T |
| 180 | CHR17 | 10649078 | T | C | T/C | C/C | C/C | T/C | C/C | T/C |
| 181 | CHR17 | 12787849 | G | T | G/T | G/T | T/T | G/T | T/T | G/T |
| 182 | CHR17 | 29928726 | A | C | A/C | A/A | A/C | A/C | A/C | A/A |
| 183 | CHR17 | 33518961 | T | C | T/C | C/C | T/C | T/T | T/T | T/T |
| 184 | CHR17 | 34335677 | C | T | C/C | C/T | C/T | C/T | T/T | T/T |
| 185 | CHR17 | 34462308 | T | C | C/C | C/C | C/C | T/T | T/C | C/C |
| 186 | CHR17 | 37408338 | T | C | C/C | C/C | T/T | T/T | T/T | T/C |
| 187 | CHR17 | 38080619 | G | A | A/A | G/A | G/A | G/A | G/A | G/A |
| 188 | CHR17 | 39124697 | G | A | G/T | T/T | G/T | T/T | T/T | G/T |
| 189 | CHR17 | 43096822 | A | G | A/G | G/G | G/G | G/G | G/G | A/A |
| 190 | CHR17 | 54117112 | G | A | G/G | G/G | G/A | G/A | G/G | G/A |
| 191 | CHR17 | 57371669 | T | C | T/C | T/C | C/C | T/C | T/C | C/C |
| 192 | CHR17 | 61882767 | C | T | T/T | T/T | T/T | T/T | T/T | T/T |

TABLE 19
Chromosome 18

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 193 | CHR18 | 6745949 | A | G | A/G | A/A | A/G | A/G | A/G | A/G |
| 194 | CHR18 | 10189759 | A | G | A/G | G/G | G/G | A/G | G/G | A/G |
| 195 | CHR18 | 11396609 | G | A | G/A | G/A | G/A | G/G | G/A | G/A |
| 196 | CHR18 | 14626636 | G | C | G/C | G/C | C/C | G/G | G/C | G/C |
| 197 | CHR18 | 16385020 | T | C | T/C | T/C | C/C | T/C | C/C | T/T |
| 198 | CHR18 | 16388978 | A | C | A/C | A/C | C/C | A/C | C/C | A/A |
| 199 | CHR18 | 16823595 | T | A | A/A | T/A | T/A | A/A | T/A | T/T |
| 200 | CHR18 | 31579269 | C | T | C/C | C/C | C/C | C/T | C/C | C/C |
| 201 | CHR18 | 44737897 | C | G | C/C | C/C | G/G | G/G | G/G | C/G |
| 202 | CHR18 | 44745010 | T | A | A/A | A/A | T/T | T/T | T/T | T/A |
| 203 | CHR18 | 47325586 | A | G | A/A | A/G | A/A | G/G | A/G | G/G |
| 204 | CHR18 | 48695616 | T | C | T/C | T/C | T/C | T/T | T/T | T/T |
| 205 | CHR18 | 54361347 | A | G | A/A | A/G | G/G | G/G | G/G | A/G |
| 206 | CHR18 | 55614071 | A | T | A/A | A/A | A/T | A/T | A/T | A/T |

TABLE 20
Chromosome 19

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 207 | CHR19 | 841347 | A | G | A/A | A/A | A/A | A/A | A/A | A/A |
| 208 | CHR19 | 15926130 | A | C | C/C | A/C | A/C | C/C | A/C | C/C |
| 209 | CHR19 | 27288167 | T | G | G/G | G/G | G/G | G/G | G/G | T/G |
| 210 | CHR19 | 30246414 | T | C | T/C | T/T | T/C | T/C | C/C | T/C |
| 211 | CHR19 | 40189405 | C | A | C/A | C/A | A/A | C/A | C/A | C/A |
| 212 | CHR19 | 42756283 | T | C | T/T | T/T | T/C | C/C | T/C | T/T |
| 213 | CHR19 | 46293046 | G | T | G/G | G/G | G/G | G/T | G/T | G/T |
| 214 | CHR19 | 47470564 | C | A | C/C | C/C | C/C | C/A | C/A | C/A |

TABLE 21
Chromosome 20

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 215 | CHR20 | 2994432 | C | G | C/C | C/G | C/G | C/C | C/G | C/G |
| 216 | CHR20 | 3713321 | T | A | T/T | T/T | T/A | T/A | A/A | T/T |
| 217 | CHR20 | 6046176 | T | C | T/C | T/C | T/T | T/C | T/T | T/C |
| 218 | CHR20 | 10797073 | G | A | G/G | G/A | G/A | G/G | G/A | G/A |
| 219 | CHR20 | 13740894 | C | T | C/T | C/T | C/T | C/C | C/T | C/T |
| 220 | CHR20 | 43362676 | T | C | T/T | T/C | T/T | T/T | T/T | T/T |
| 221 | CHR20 | 45777531 | T | C | T/C | T/T | C/C | C/C | C/C | T/T |
| 222 | CHR20 | 48602465 | G | A | G/A | G/A | G/G | G/G | G/G | A/A |
| 223 | CHR20 | 49900586 | C | T | C/C | C/C | C/T | C/T | C/T | C/C |
| 224 | CHR20 | 57167714 | G | A | G/G | G/A | G/A | G/A | G/A | A/A |

TABLE 22
Chromosome 21

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 225 | CHR21 | 5052079 | C | T | C/C | C/C | C/C | C/C | C/C | C/C |
| 226 | CHR21 | 14317679 | C | T | T/T | T/T | T/T | C/T | C/T | C/T |
| 227 | CHR21 | 15558670 | C | T | C/T | C/T | C/C | C/C | C/C | C/C |
| 228 | CHR21 | 22581321 | G | A | G/G | G/A | A/A | A/A | A/A | G/A |
| 229 | CHR21 | 25537675 | C | T | C/T | C/C | C/T | C/T | T/T | C/C |
| 230 | CHR21 | 29796784 | G | A | G/G | G/A | A/A | A/A | A/A | G/A |
| 231 | CHR21 | 31751817 | T | C | T/C | C/C | T/C | T/C | C/C | T/C |
| 232 | CHR21 | 35719434 | C | T | C/T | C/T | C/T | C/T | C/C | T/T |
| 233 | CHR21 | 37997682 | C | T | C/T | C/T | T/T | T/T | T/T | C/C |
| 234 | CHR21 | 46176267 | T | G | T/T | T/T | T/T | T/G | T/T | T/T |

TABLE 23
Chromosome 22

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 235 | CHR22 | 641125 | T | C | T/T | T/T | C/C | T/C | C/C | T/C |
| 236 | CHR22 | 26694580 | A | G | A/G | G/G | A/G | A/A | A/A | A/G |
| 237 | CHR22 | 33594175 | G | A | G/G | G/A | G/G | G/A | G/G | G/G |
| 238 | CHR22 | 33934047 | C | T | T/T | C/T | C/T | C/T | C/C | C/T |
| 239 | CHR22 | 37522364 | A | G | A/A | A/A | A/G | A/G | A/A | A/A |
| 240 | CHR22 | 39647748 | T | C | T/C | C/C | T/C | T/C | T/T | T/C |
| 241 | CHR22 | 42162642 | C | G | C/C | C/C | C/G | C/G | C/C | C/G |
| 242 | CHR22 | 55308193 | C | A | C/C | C/A | C/C | A/A | C/A | A/A |
| 243 | CHR22 | 58143110 | A | T | A/T | A/A | A/A | A/A | A/A | A/A |
| 244 | CHR22 | 61153661 | A | G | A/G | A/G | A/G | A/G | G/G | A/A |

TABLE 24
Chromosome 23

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 245 | CHR23 | 2895637 | C | A | A/A | C/A | C/C | C/A | C/A | C/A |
| 246 | CHR23 | 18407255 | T | C | T/C | C/C | T/C | T/C | C/C | T/T |
| 247 | CHR23 | 42886681 | C | A | C/A | C/A | C/C | C/C | C/C | C/A |
| 248 | CHR23 | 44497217 | C | T | C/C | C/T | C/T | C/T | C/T | C/T |
| 249 | CHR23 | 46719873 | A | C | A/A | A/A | A/C | A/C | A/C | A/A |
| 250 | CHR23 | 48055836 | G | T | T/T | G/T | G/G | G/G | G/G | G/G |
| 251 | CHR23 | 50772488 | T | C | T/C | C/C | T/C | C/C | C/C | T/C |
| 252 | CHR23 | 52286800 | C | T | T/T | T/T | C/T | C/T | C/T | C/T |

TABLE 25
Chromosome 24

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 253 | CHR24 | 3805961 | G | A | A/A | G/A | A/A | A/A | A/A | A/A |
| 254 | CHR24 | 18599997 | T | C | T/C | C/C | C/C | C/C | C/C | T/C |
| 255 | CHR24 | 23393510 | T | G | G/G | G/G | G/G | G/G | G/G | T/G |
| 256 | CHR24 | 23393552 | C | T | C/C | C/C | C/C | C/C | C/C | C/C |
| 257 | CHR24 | 27925354 | G | A | A/A | A/A | G/A | A/A | G/A | G/A |
| 258 | CHR24 | 29909901 | T | C | T/T | T/T | T/C | T/T | T/T | T/T |
| 259 | CHR24 | 30954773 | A | G | A/G | A/G | A/G | A/A | A/A | A/G |
| 260 | CHR24 | 33945617 | A | C | A/A | A/A | A/A | A/A | A/A | A/C |
| 261 | CHR24 | 43589304 | A | G | A/G | A/G | A/G | A/G | A/A | A/A |
| 262 | CHR24 | 45191477 | A | G | A/G | G/G | A/G | A/G | A/A | A/A |
| 263 | CHR24 | 45464813 | T | G | T/T | T/T | T/T | T/T | G/G | G/G |
| 264 | CHR24 | 47381908 | A | C | A/A | A/A | G/G | A/A | A/A | A/A |

TABLE 26
Chromosome 25

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 265 | CHR25 | 2073511 | G | T | G/T | T/T | G/G | G/G | G/G | G/T |
| 266 | CHR25 | 4614777 | A | G | A/G | A/G | A/G | A/G | A/G | A/G |
| 267 | CHR25 | 8834951 | G | C | G/G | G/G | G/G | G/G | G/G | G/G |
| 268 | CHR25 | 13507345 | G | T | G/G | G/T | G/T | G/G | G/G | G/G |
| 269 | CHR25 | 18601807 | G | C | G/C | G/G | C/C | C/C | C/C | G/G |
| 270 | CHR25 | 29670795 | G | T | G/G | G/G | T/T | T/T | T/T | T/T |
| 271 | CHR25 | 33986348 | T | C | T/T | T/T | C/C | C/C | C/C | T/T |
| 272 | CHR25 | 37575208 | G | A | G/A | G/A | G/A | G/A | G/A | G/A |
| 273 | CHR25 | 47708600 | G | A | A/A | G/A | G/G | G/G | G/G | G/A |
| 274 | CHR25 | 48221181 | C | T | C/C | C/C | C/C | C/C | C/C | C/C |

TABLE 27
Chromosome 26

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 275 | CHR26 | 16198323 | A | C | A/C | C/C | C/C | C/C | C/C | C/C |
| 276 | CHR26 | 24271280 | G | C | G/C | G/G | G/C | C/C | C/C | C/C |
| 277 | CHR26 | 29366164 | A | G | A/G | A/A | A/A | A/A | A/A | A/A |
| 278 | CHR26 | 35071515 | G | A | A/A | A/A | A/A | A/A | A/A | G/G |
| 279 | CHR26 | 35779460 | C | A | C/A | C/C | C/A | C/A | C/C | C/A |
| 280 | CHR26 | 36214508 | G | A | G/G | Nocall | G/G | G/G | G/G | G/G |

TABLE 28
Chromosome 27

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 281 | CHR27 | 2479833 | C | T | C/C | C/C | C/C | C/C | C/C | C/C |
| 282 | CHR27 | 2619058 | G | A | G/A | G/G | G/A | G/G | G/A | G/A |
| 283 | CHR27 | 20948372 | G | A | G/A | G/A | G/A | A/A | A/A | G/G |
| 284 | CHR27 | 22599860 | T | C | T/C | T/T | C/C | T/C | T/C | T/C |
| 285 | CHR27 | 26257754 | G | A | G/G | G/G | G/A | G/A | A/A | G/G |
| 286 | CHR27 | 34444177 | C | T | C/T | C/T | C/T | T/T | C/T | C/T |
| 287 | CHR27 | 41049333 | T | G | T/T | T/G | T/T | T/G | T/G | G/G |
| 288 | CHR27 | 42526114 | G | A | G/G | G/G | G/G | G/A | G/A | G/G |
| 289 | CHR27 | 44650848 | A | C | A/A | A/A | A/A | A/A | A/A | C/C |

TABLE 29
Chromosome 28

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 290 | CHR28 | 9703418 | C | T | C/T | C/T | C/T | C/T | C/T | C/T |
| 291 | CHR28 | 9877730 | A | G | A/G | A/G | G/G | G/G | G/G | A/G |
| 292 | CHR28 | 12804225 | C | T | C/C | C/C | T/T | C/T | T/T | C/T |
| 293 | CHR28 | 18509221 | C | T | C/C | C/C | T/T | T/T | T/T | C/C |
| 294 | CHR28 | 22387315 | G | A | G/A | G/A | G/G | G/G | G/G | G/G |
| 295 | CHR28 | 27652746 | C | A | C/C | C/C | C/C | C/C | C/C | C/C |
| 296 | CHR28 | 34478533 | A | G | A/A | A/G | G/G | A/G | G/G | A/A |
| 297 | CHR28 | 35104850 | G | A | G/A | G/A | G/A | G/A | G/A | G/G |
| 298 | CHR28 | 38885325 | C | T | T/T | T/T | T/T | T/T | T/T | C/T |
| 299 | CHR28 | 39326607 | C | T | C/C | C/C | C/C | C/C | C/C | C/T |

TABLE 30
Chromosome 29

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 300 | CHR29 | 251970 | A | G | G/G | A/G | A/G | A/A | A/G | A/A |
| 301 | CHR29 | 963602 | G | C | G/G | G/G | G/G | G/G | G/G | G/G |
| 302 | CHR29 | 4020192 | A | G | A/G | G/G | G/G | G/G | G/G | G/G |
| 303 | CHR29 | 4022252 | C | T | C/C | C/T | T/T | T/T | T/T | T/T |
| 304 | CHR29 | 9625359 | G | A | A/A | G/A | G/G | G/G | G/G | G/A |
| 305 | CHR29 | 19681270 | A | G | A/G | A/G | A/G | A/A | A/A | G/G |
| 306 | CHR29 | 22992304 | G | A | G/A | G/G | A/A | G/A | G/A | A/A |
| 307 | CHR29 | 36319325 | T | G | G/G | G/G | T/T | T/T | T/T | T/G |

TABLE 31
Chromosome 30

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 308 | CHR30 | 136521 | C | T | C/T | C/T | C/T | C/T | C/T | C/C |
| 309 | CHR30 | 3896482 | T | C | C/C | C/C | T/C | T/T | T/C | C/C |
| 310 | CHR30 | 10012939 | T | C | C/C | C/C | T/T | T/T | T/T | T/T |
| 311 | CHR30 | 11735245 | C | T | T/T | T/T | C/C | C/C | C/C | T/T |
| 312 | CHR30 | 15542105 | A | G | G/G | A/G | G/G | G/G | G/G | A/G |
| 313 | CHR30 | 27619023 | T | C | T/C | T/C | C/C | T/C | C/C | T/T |
| 314 | CHR30 | 32852404 | C | T | C/C | C/T | C/T | T/T | C/T | C/C |
| 315 | CHR30 | 39147226 | G | T | G/T | T/T | G/T | G/T | G/T | G/G |

TABLE 32
Chromosome 31

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 316 | CHR31 | 20912553 | G | A | A/A | A/A | G/G | G/G | G/G | G/A |
| 317 | CHR31 | 21068798 | C | T | C/T | T/T | C/T | C/T | C/C | T/T |
| 318 | CHR31 | 26409294 | A | T | A/T | T/T | A/T | A/T | A/T | A/A |
| 319 | CHR31 | 36234234 | C | T | C/C | C/T | T/T | T/T | T/T | T/T |
| 320 | CHR31 | 39391935 | G | A | A/A | G/A | G/A | G/A | G/A | A/A |

TABLE 33
Chromosome 32

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 321 | CHR32 | 679380 | G | A | G/G | G/G | G/G | G/A | G/G | G/A |
| 322 | CHR32 | 4509367 | G | T | G/T | G/T | G/G | G/G | G/G | G/G |
| 323 | CHR32 | 5231894 | C | A | C/A | A/A | C/C | C/C | C/C | C/C |
| 324 | CHR32 | 13183511 | A | G | A/G | G/G | G/G | G/G | G/G | A/A |
| 325 | CHR32 | 17792284 | C | T | C/T | C/C | C/T | C/T | C/C | C/T |
| 326 | CHR32 | 22033358 | G | A | G/A | G/G | G/G | G/G | G/G | G/G |
| 327 | CHR32 | 32382778 | A | G | A/A | A/G | G/G | A/G | A/G | G/G |
| 328 | CHR32 | 32401459 | T | C | T/T | T/C | C/C | T/C | T/C | C/C |
| 329 | CHR32 | 38020426 | C | T | C/C | C/C | C/C | C/C | C/C | C/C |

TABLE 34
Chromosome 33

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 330 | CHR33 | 5958606 | C | G | C/G | C/G | C/C | C/C | C/C | C/C |
| 331 | CHR33 | 15018500 | C | T | C/T | C/C | C/C | C/C | C/C | C/C |
| 332 | CHR33 | 15233992 | G | A | G/A | A/A | G/G | G/G | G/G | A/A |
| 333 | CHR33 | 22472901 | C | A | C/A | C/A | C/A | C/A | C/A | C/C |
| 334 | CHR33 | 22648231 | T | C | T/C | T/T | T/T | T/T | T/T | T/C |
| 335 | CHR33 | 23742061 | A | G | G/G | G/G | A/G | A/G | A/G | G/G |
| 336 | CHR33 | 30777707 | G | A | G/G | G/G | G/G | G/A | G/G | G/G |

TABLE 35
Chromosome 34

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 337 | CHR34 | 195313 | C | A | C/A | C/A | C/A | C/C | C/C | C/C |
| 338 | CHR34 | 11079546 | A | G | A/A | A/A | A/A | A/A | A/A | A/A |
| 339 | CHR34 | 11211095 | T | A | A/A | A/A | A/A | A/A | A/A | T/A |
| 340 | CHR34 | 24351570 | C | T | T/T | T/T | C/T | C/C | C/C | C/T |
| 341 | CHR34 | 24396298 | A | G | A/A | A/G | A/G | A/A | A/G | G/G |
| 342 | CHR34 | 32345428 | C | G | C/C | C/G | C/G | G/G | G/G | C/G |
| 343 | CHR34 | 34993916 | A | G | A/A | A/A | G/G | A/A | A/G | G/G |
| 344 | CHR34 | 37323213 | T | C | T/C | T/T | T/T | C/C | T/C | T/C |
| 345 | CHR34 | 37730426 | T | C | T/C | T/C | T/T | T/T | T/T | T/T |
| 346 | CHR34 | 41672975 | T | A | A/A | A/A | A/A | T/A | A/A | T/T |
| 347 | CHR34 | 41703614 | G | A | G/A | G/G | G/G | A/A | G/A | G/A |

TABLE 36
Chromosome 35

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 348 | CHR35 | 58695 | A | G | A/A | A/A | A/A | A/A | A/A | A/A |
| 349 | CHR35 | 13480334 | C | T | C/C | C/C | C/C | C/C | C/C | C/T |
| 350 | CHR35 | 15283717 | G | A | G/G | G/G | G/G | G/G | G/G | G/G |
| 351 | CHR35 | 15345329 | C | A | C/C | C/C | C/C | C/C | C/C | C/A |
| 352 | CHR35 | 22561060 | C | G | C/G | C/G | G/G | Nocall | Nocall | G/G |
| 353 | CHR35 | 26510300 | G | T | T/T | T/T | T/T | T/T | T/T | T/T |

TABLE 37
Chromosome 36

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 354 | CHR36 | 10700 | A | G | A/A | A/A | A/G | A/G | G/G | A/G |
| 355 | CHR36 | 288045 | A | G | A/A | A/A | A/G | G/G | A/G | A/G |
| 356 | CHR36 | 3565500 | A | G | A/G | A/A | A/G | A/G | A/A | A/G |
| 357 | CHR36 | 9241262 | G | A | G/G | G/G | A/A | A/A | A/A | G/G |
| 358 | CHR36 | 10084888 | C | T | C/C | C/T | C/T | C/T | C/C | T/T |
| 359 | CHR36 | 12714421 | A | G | A/A | A/A | A/G | A/G | G/G | A/G |
| 360 | CHR36 | 12723744 | A | C | A/A | A/A | A/C | A/C | C/C | A/C |
| 361 | CHR36 | 16424099 | G | T | G/G | G/G | G/G | G/G | G/G | G/T |
| 362 | CHR36 | 18627936 | A | G | A/G | G/G | G/G | G/G | G/G | G/G |
| 363 | CHR36 | 23459390 | G | A | G/G | G/G | G/A | G/A | G/A | G/A |

TABLE 38
Chromosome 37

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 364 | CHR37 | 9398945 | G | A | G/A | G/A | G/A | G/A | G/G | G/A |
| 365 | CHR37 | 15436615 | T | C | T/C | T/C | C/C | C/C | C/C | T/C |
| 366 | CHR37 | 18338930 | C | A | C/A | A/A | C/C | C/C | C/C | C/A |
| 367 | CHR37 | 26611359 | C | T | C/T | C/T | C/C | C/C | C/C | C/C |
| 368 | CHR37 | 27667297 | C | T | C/C | C/C | C/T | C/C | C/C | C/T |
| 369 | CHR37 | 28611801 | A | G | A/G | G/G | A/G | G/G | G/G | G/G |
| 370 | CHR37 | 30110473 | T | C | T/C | C/C | C/C | C/C | C/C | T/C |
| 371 | CHR37 | 30902202 | T | C | T/C | T/C | T/T | T/T | T/T | C/C |

TABLE 39
Chromosome 38

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 372 | CHR38 | 9224942 | T | G | G/G | T/G | T/G | G/G | G/G | T/T |
| 373 | CHR38 | 13098194 | A | C | C/C | C/C | A/A | C/C | A/C | C/C |
| 374 | CHR38 | 15271384 | A | G | A/A | A/G | G/G | A/A | A/G | A/A |
| 375 | CHR38 | 17507522 | C | T | C/C | C/C | C/C | C/C | C/C | C/C |
| 376 | CHR38 | 17657161 | T | C | C/C | T/C | T/C | C/C | T/C | C/C |
| 377 | CHR38 | 19172567 | C | A | A/A | C/A | C/A | C/A | C/A | C/A |
| 378 | CHR38 | 20441216 | T | C | T/T | T/C | C/C | T/C | T/C | T/C |
| 379 | CHR38 | 20930997 | C | A | A/A | A/A | A/A | A/A | A/A | C/A |
| 380 | CHR38 | 23702640 | G | C | C/C | C/C | C/C | C/C | C/C | G/C |

TABLE 40
X chromosome

| | | | | | | | Beagle | Beagle | Beagle | |
|---|---|---|---|---|---|---|---|---|---|---|
| No. | CHROM | POS | Ref | Alt | Gyeomdung | Lotto | Mom | Dad | Daughter | Mungchi |
| 381 | CHRX | 4245861 | G | A | G/G | G/G | G/G | G/G | G/G | G/G |

TABLE 41

| No. | CHROM | POS | Ref | Alt |
|---|---|---|---|---|
| 1 | CHR1 | 3962719 | A | G |
| 2 | CHR1 | 12076349 | C | T |
| 3 | CHR1 | 20842130 | C | T |
| 4 | CHR1 | 44484310 | T | C |
| 5 | CHR1 | 54192143 | G | C |
| 6 | CHR1 | 69171090 | A | G |
| 7 | CHR1 | 70238933 | C | T |
| 8 | CHR1 | 72613047 | T | C |
| 9 | CHR1 | 74450772 | G | A |
| 10 | CHR1 | 80971770 | A | G |
| 11 | CHR1 | 105514131 | G | C |
| 12 | CHR1 | 106430955 | G | A |
| 13 | CHR1 | 117495849 | C | A |
| 14 | CHR1 | 119306331 | A | G |
| 15 | CHR1 | 119414584 | G | A |
| 16 | CHR2 | 2610859 | T | C |
| 17 | CHR2 | 38293797 | A | G |
| 18 | CHR2 | 67888039 | A | G |
| 19 | CHR2 | 77806065 | G | A |
| 20 | CHR2 | 84535676 | G | A |
| 21 | CHR3 | 1252765 | C | A |
| 22 | CHR3 | 10255068 | C | T |
| 23 | CHR3 | 24757939 | G | A |
| 24 | CHR3 | 28054239 | G | A |
| 25 | CHR3 | 37849557 | C | T |
| 26 | CHR3 | 40302288 | A | G |
| 27 | CHR3 | 41849479 | G | A |
| 28 | CHR3 | 43055696 | A | G |
| 29 | CHR3 | 43063677 | T | C |
| 30 | CHR3 | 44126666 | G | C |
| 31 | CHR3 | 60621051 | T | C |
| 32 | CHR3 | 64084413 | G | A |
| 33 | CHR3 | 73570828 | T | C |
| 34 | CHR3 | 90291255 | T | C |
| 35 | CHR3 | 91103945 | G | T |
| 36 | CHR3 | 91626907 | C | T |
| 37 | CHR4 | 10582893 | A | T |
| 38 | CHR4 | 31301072 | G | A |
| 39 | CHR4 | 39182836 | A | T |
| 40 | CHR4 | 42104780 | T | C |
| 41 | CHR4 | 53911120 | G | C |
| 42 | CHR4 | 64121754 | T | C |
| 43 | CHR4 | 67040898 | C | T |
| 44 | CHR4 | 67040939 | G | A |
| 45 | CHR4 | 70217695 | G | A |
| 46 | CHR4 | 75910211 | A | G |
| 47 | CHR4 | 86049027 | C | T |
| 48 | CHR4 | 86998873 | C | T |
| 49 | CHR5 | 5410890 | C | T |
| 50 | CHR5 | 13080303 | A | G |
| 51 | CHR5 | 25193842 | G | A |
| 52 | CHR5 | 26320165 | C | T |
| 53 | CHR5 | 36642434 | A | G |
| 54 | CHR5 | 44642087 | A | T |
| 55 | CHR5 | 44650576 | G | A |
| 56 | CHR5 | 50794117 | G | C |
| 57 | CHR5 | 55349573 | A | G |
| 58 | CHR5 | 56989849 | A | G |
| 59 | CHR5 | 63694334 | G | A |
| 60 | CHR5 | 63694460 | C | T |
| 61 | CHR5 | 64611038 | G | A |
| 62 | CHR5 | 79189887 | C | A |
| 63 | CHR5 | 85451804 | A | G |
| 64 | CHR5 | 87925312 | A | G |
| 65 | CHR5 | 88910280 | T | C |
| 66 | CHR6 | 11553458 | A | G |
| 67 | CHR6 | 16653546 | G | C |
| 68 | CHR6 | 33976751 | T | C |
| 69 | CHR6 | 44672748 | A | G |
| 70 | CHR6 | 64006720 | A | G |
| 71 | CHR6 | 64403024 | G | A |
| 72 | CHR6 | 74727390 | G | C |
| 73 | CHR7 | 76294 | A | G |
| 74 | CHR7 | 3318809 | A | G |
| 75 | CHR7 | 6423299 | C | T |
| 76 | CHR7 | 15011628 | A | G |
| 77 | CHR7 | 15017979 | G | A |
| 78 | CHR7 | 36171712 | T | C |
| 79 | CHR7 | 36555518 | T | C |
| 80 | CHR7 | 38350972 | C | G |
| 81 | CHR7 | 46413871 | C | T |
| 82 | CHR7 | 63919631 | G | C |
| 83 | CHR7 | 76487265 | G | A |
| 84 | CHR7 | 80017081 | G | A |
| 85 | CHR7 | 80443374 | A | G |
| 86 | CHR8 | 5291824 | A | G |
| 87 | CHR8 | 6188937 | G | A |
| 88 | CHR8 | 6267365 | G | T |
| 89 | CHR8 | 10044405 | C | T |
| 90 | CHR8 | 10413839 | G | A |
| 91 | CHR8 | 18121580 | C | T |
| 92 | CHR8 | 19076567 | T | C |
| 93 | CHR8 | 24614720 | G | A |
| 94 | CHR8 | 41635950 | T | A |
| 95 | CHR8 | 45852939 | A | G |
| 96 | CHR8 | 52381322 | G | A |
| 97 | CHR8 | 63196958 | G | A |
| 98 | CHR8 | 67183794 | C | T |
| 99 | CHR8 | 71834501 | G | C |
| 100 | CHR9 | 10346478 | G | A |
| 101 | CHR9 | 20867959 | C | T |
| 102 | CHR9 | 22610227 | A | G |
| 103 | CHR9 | 32506288 | G | A |
| 104 | CHR9 | 40096141 | T | C |
| 105 | CHR9 | 40702757 | T | C |
| 106 | CHR9 | 50114927 | G | A |
| 107 | CHR9 | 52710991 | C | T |
| 108 | CHR9 | 56021221 | A | G |
| 109 | CHR9 | 60437147 | A | G |
| 110 | CHR10 | 8085469 | C | T |
| 111 | CHR10 | 8183593 | C | T |
| 112 | CHR10 | 10652659 | C | T |
| 113 | CHR10 | 14685262 | C | T |
| 114 | CHR10 | 15113284 | T | A |
| 115 | CHR10 | 15362456 | A | G |
| 116 | CHR10 | 22409408 | A | G |
| 117 | CHR10 | 30034450 | A | G |
| 118 | CHR10 | 35942926 | T | C |
| 119 | CHR10 | 39548483 | C | T |
| 120 | CHR10 | 47923623 | C | T |
| 121 | CHR10 | 49188334 | C | T |
| 122 | CHR10 | 57954366 | C | T |
| 123 | CHR10 | 66922269 | T | C |
| 124 | CHR10 | 67126524 | G | A |
| 125 | CHR10 | 68918209 | C | T |
| 126 | CHR11 | 1161870 | G | A |
| 127 | CHR11 | 3242754 | A | C |
| 128 | CHR11 | 5318488 | C | T |
| 129 | CHR11 | 12479869 | C | T |
| 130 | CHR11 | 23907101 | A | C |
| 131 | CHR11 | 33317810 | T | A |
| 132 | CHR11 | 33326685 | C | T |
| 133 | CHR11 | 33326726 | CCT | — |
| 134 | CHR11 | 62157625 | G | A |
| 135 | CHR11 | 65603333 | C | A |
| 136 | CHR11 | 70698603 | G | T |
| 137 | CHR11 | 72333665 | G | C |
| 138 | CHR11 | 73866275 | C | G |
| 139 | CHR12 | 186569 | C | G |
| 140 | CHR12 | 5579055 | A | G |
| 141 | CHR12 | 8532712 | A | G |
| 142 | CHR12 | 10683432 | T | G |
| 143 | CHR12 | 23059939 | A | G |
| 144 | CHR12 | 35306641 | A | G |
| 145 | CHR12 | 40681020 | G | A |
| 146 | CHR12 | 55201839 | G | A |
| 147 | CHR12 | 68125319 | A | G |
| 148 | CHR12 | 70657733 | G | A |
| 149 | CHR12 | 72440787 | A | G |
| 150 | CHR13 | 2561486 | C | T |
| 151 | CHR13 | 7791160 | T | A |
| 152 | CHR13 | 8704192 | T | C |
| 153 | CHR13 | 40584681 | G | C |
| 154 | CHR13 | 40616856 | T | C |
| 155 | CHR13 | 44869343 | T | G |
| 156 | CHR13 | 59896033 | T | G |
| 157 | CHR13 | 62898269 | G | A |
| 158 | CHR14 | 7319133 | C | A |
| 159 | CHR14 | 13726593 | ATCT | — |
| 160 | CHR14 | 21072223 | C | T |
| 161 | CHR14 | 25812525 | C | T |
| 162 | CHR14 | 43370655 | A | G |
| 163 | CHR14 | 50063321 | A | G |
| 164 | CHR14 | 55735620 | C | T |
| 165 | CHR14 | 58465266 | A | G |
| 166 | CHR15 | 1165219 | C | T |
| 167 | CHR15 | 19299365 | T | C |
| 168 | CHR15 | 22834903 | C | A |
| 169 | CHR15 | 31936820 | T | A |
| 170 | CHR15 | 61796402 | G | T |
| 171 | CHR16 | 29634940 | T | C |
| 172 | CHR16 | 29675662 | C | A |
| 173 | CHR16 | 38237959 | A | T |
| 174 | CHR16 | 46884446 | T | G |
| 175 | CHR16 | 57958947 | C | T |
| 176 | CHR16 | 58093031 | T | G |
| 177 | CHR17 | 7140289 | G | C |
| 178 | CHR17 | 9407683 | G | A |
| 179 | CHR17 | 10050848 | T | C |
| 180 | CHR17 | 10649078 | T | C |
| 181 | CHR17 | 12787849 | G | T |
| 182 | CHR17 | 29928726 | A | C |
| 183 | CHR17 | 33518961 | T | C |
| 184 | CHR17 | 34335677 | C | T |
| 185 | CHR17 | 34462308 | T | C |
| 186 | CHR17 | 37408338 | T | C |
| 187 | CHR17 | 38080619 | G | A |
| 188 | CHR17 | 39124697 | G | A |
| 189 | CHR17 | 43096822 | A | G |
| 190 | CHR17 | 54117112 | G | A |
| 191 | CHR17 | 57371669 | T | C |
| 192 | CHR17 | 61882767 | C | T |
| 193 | CHR18 | 6745949 | A | G |
| 194 | CHR18 | 10189759 | A | G |
| 195 | CHR18 | 11396609 | G | A |
| 196 | CHR18 | 14626636 | G | C |
| 197 | CHR18 | 16385020 | T | C |
| 198 | CHR18 | 16388978 | A | C |
| 199 | CHR18 | 16823595 | T | A |
| 200 | CHR18 | 31579269 | C | T |
| 201 | CHR18 | 44737897 | C | G |
| 202 | CHR18 | 44745010 | T | A |
| 203 | CHR18 | 47325586 | A | G |
| 204 | CHR18 | 48695616 | T | C |
| 205 | CHR18 | 54361347 | A | G |
| 206 | CHR18 | 55614071 | A | T |
| 207 | CHR19 | 841347 | A | G |
| 208 | CHR19 | 15926130 | A | C |
| 209 | CHR19 | 27288167 | T | G |
| 210 | CHR19 | 30246414 | T | C |
| 211 | CHR19 | 40189405 | C | A |
| 212 | CHR19 | 42756283 | T | C |
| 213 | CHR19 | 46293046 | G | T |
| 214 | CHR19 | 47470564 | C | A |
| 215 | CHR20 | 2994432 | C | G |
| 216 | CHR20 | 3713321 | T | A |
| 217 | CHR20 | 6046176 | T | C |
| 218 | CHR20 | 10797073 | G | A |
| 219 | CHR20 | 13740894 | C | T |
| 220 | CHR20 | 43362676 | T | C |
| 221 | CHR20 | 45777531 | T | C |
| 222 | CHR20 | 48602465 | G | A |
| 223 | CHR20 | 49900586 | C | T |
| 224 | CHR20 | 57167714 | G | A |
| 225 | CHR21 | 5052079 | C | T |
| 226 | CHR21 | 14317679 | C | T |
| 227 | CHR21 | 15558670 | C | T |
| 228 | CHR21 | 22581321 | G | A |
| 229 | CHR21 | 25537675 | C | T |
| 230 | CHR21 | 29796784 | G | A |
| 231 | CHR21 | 31751817 | T | C |
| 232 | CHR21 | 35719434 | C | T |
| 233 | CHR21 | 37997682 | C | T |
| 234 | CHR21 | 46176267 | T | G |
| 235 | CHR22 | 641125 | T | C |
| 236 | CHR22 | 26694580 | A | G |
| 237 | CHR22 | 33594175 | G | A |
| 238 | CHR22 | 33934047 | C | T |
| 239 | CHR22 | 37522364 | A | G |
| 240 | CHR22 | 39647748 | T | C |
| 241 | CHR22 | 42162642 | C | G |
| 242 | CHR22 | 55308193 | C | A |
| 243 | CHR22 | 58143110 | A | T |
| 244 | CHR22 | 61153661 | A | G |
| 245 | CHR23 | 2895637 | C | A |
| 246 | CHR23 | 18407255 | T | C |
| 247 | CHR23 | 42886681 | C | A |
| 248 | CHR23 | 44497217 | C | T |
| 249 | CHR23 | 46719873 | A | C |
| 250 | CHR23 | 48055836 | G | T |
| 251 | CHR23 | 50772488 | T | C |
| 252 | CHR23 | 52286800 | C | T |
| 253 | CHR24 | 3805961 | G | A |
| 254 | CHR24 | 18599997 | T | C |
| 255 | CHR24 | 23393510 | T | G |
| 256 | CHR24 | 23393552 | C | T |
| 257 | CHR24 | 27925354 | G | A |
| 258 | CHR24 | 29909901 | T | C |
| 259 | CHR24 | 30954773 | A | G |
| 260 | CHR24 | 33945617 | A | C |
| 261 | CHR24 | 43589304 | A | G |
| 262 | CHR24 | 45191477 | A | G |
| 263 | CHR24 | 45464813 | T | G |
| 264 | CHR24 | 47381908 | A | C |
| 265 | CHR25 | 2073511 | G | T |
| 266 | CHR25 | 4614777 | A | G |
| 267 | CHR25 | 8834951 | G | C |
| 268 | CHR25 | 13507345 | G | T |
| 269 | CHR25 | 18601807 | G | C |
| 270 | CHR25 | 29670795 | G | T |
| 271 | CHR25 | 33986348 | T | C |
| 272 | CHR25 | 37575208 | G | A |
| 273 | CHR25 | 47708600 | G | A |
| 274 | CHR25 | 48221181 | C | T |
| 275 | CHR26 | 16198323 | A | C |
| 276 | CHR26 | 24271280 | G | C |
| 277 | CHR26 | 29366164 | A | G |
| 278 | CHR26 | 35071515 | G | A |
| 279 | CHR26 | 35779460 | C | A |
| 280 | CHR26 | 36214508 | G | A |
| 281 | CHR27 | 2479833 | C | T |
| 282 | CHR27 | 2619058 | G | A |
| 283 | CHR27 | 20948372 | G | A |
| 284 | CHR27 | 22599860 | T | C |
| 285 | CHR27 | 26257754 | G | A |
| 286 | CHR27 | 34444177 | C | T |
| 287 | CHR27 | 41049333 | T | G |
| 288 | CHR27 | 42526114 | G | A |
| 289 | CHR27 | 44650848 | A | C |
| 290 | CHR28 | 9703418 | C | T |
| 291 | CHR28 | 9877730 | A | G |
| 292 | CHR28 | 12804225 | C | T |
| 293 | CHR28 | 18509221 | C | T |
| 294 | CHR28 | 22387315 | G | A |
| 295 | CHR28 | 27652746 | C | A |
| 296 | CHR28 | 34478533 | A | G |
| 297 | CHR28 | 35104850 | G | A |
| 298 | CHR28 | 38885325 | C | T |
| 299 | CHR28 | 39326607 | C | T |
| 300 | CHR29 | 251970 | A | G |
| 301 | CHR29 | 963602 | G | C |
| 302 | CHR29 | 4020192 | A | G |
| 303 | CHR29 | 4022252 | C | T |
| 304 | CHR29 | 9625359 | G | A |
| 305 | CHR29 | 19681270 | A | G |
| 306 | CHR29 | 22992304 | G | A |
| 307 | CHR29 | 36319325 | T | G |
| 308 | CHR30 | 136521 | C | T |
| 309 | CHR30 | 3896482 | T | C |
| 310 | CHR30 | 10012939 | T | C |
| 311 | CHR30 | 11735245 | C | T |
| 312 | CHR30 | 15542105 | A | G |
| 313 | CHR30 | 27619023 | T | C |
| 314 | CHR30 | 32852404 | C | T |
| 315 | CHR30 | 39147226 | G | T |
| 316 | CHR31 | 20912553 | G | A |
| 317 | CHR31 | 21068798 | C | T |
| 318 | CHR31 | 26409294 | A | T |
| 319 | CHR31 | 36234234 | C | T |
| 320 | CHR31 | 39391935 | G | A |
| 321 | CHR32 | 679380 | G | A |
| 322 | CHR32 | 4509367 | G | T |
| 323 | CHR32 | 5231894 | C | A |
| 324 | CHR32 | 13183511 | A | G |
| 325 | CHR32 | 17792284 | C | T |
| 326 | CHR32 | 22033358 | G | A |
| 327 | CHR32 | 32382778 | A | G |
| 328 | CHR32 | 32401459 | T | C |
| 329 | CHR32 | 38020426 | C | T |
| 330 | CHR33 | 5958606 | C | G |
| 331 | CHR33 | 15018500 | C | T |
| 332 | CHR33 | 15233992 | G | A |
| 333 | CHR33 | 22472901 | C | A |
| 334 | CHR33 | 22648231 | T | C |
| 335 | CHR33 | 23742061 | A | G |
| 336 | CHR33 | 30777707 | G | A |
| 337 | CHR34 | 195313 | C | A |
| 338 | CHR34 | 11079546 | A | G |
| 339 | CHR34 | 11211095 | T | A |
| 340 | CHR34 | 24351570 | C | T |
| 341 | CHR34 | 24396298 | A | G |
| 342 | CHR34 | 32345428 | C | G |
| 343 | CHR34 | 34993916 | A | G |
| 344 | CHR34 | 37323213 | T | C |
| 345 | CHR34 | 37730426 | T | C |
| 346 | CHR34 | 41672975 | T | A |
| 347 | CHR34 | 41703614 | G | A |
| 348 | CHR35 | 58695 | A | G |
| 349 | CHR35 | 13480334 | C | T |
| 350 | CHR35 | 15283717 | G | A |
| 351 | CHR35 | 15345329 | C | A |
| 352 | CHR35 | 22561060 | C | G |
| 353 | CHR35 | 26510300 | G | T |
| 354 | CHR36 | 10700 | A | G |
| 355 | CHR36 | 288045 | A | G |
| 356 | CHR36 | 3565500 | A | G |
| 357 | CHR36 | 9241262 | G | A |
| 358 | CHR36 | 10084888 | C | T |
| 359 | CHR36 | 12714421 | A | G |
| 360 | CHR36 | 12723744 | A | C |
| 361 | CHR36 | 16424099 | G | T |
| 362 | CHR36 | 18627936 | A | G |
| 363 | CHR36 | 23459390 | G | A |
| 364 | CHR37 | 9398945 | G | A |
| 365 | CHR37 | 15436615 | T | C |
| 366 | CHR37 | 18338930 | C | A |
| 367 | CHR37 | 26611359 | C | T |
| 368 | CHR37 | 27667297 | C | T |
| 365 | CHR37 | 28611801 | A | G |
| 370 | CHR37 | 30110473 | T | C |
| 371 | CHR37 | 30902202 | T | C |
| 372 | CHR38 | 9224942 | T | G |
| 373 | CHR38 | 13098194 | A | C |
| 374 | CHR38 | 15271384 | A | G |
| 375 | CHR38 | 17507522 | C | T |
| 376 | CHR38 | 17657161 | T | C |
| 377 | CHR38 | 19172567 | C | A |
| 378 | CHR38 | 20441216 | T | C |
| 379 | CHR38 | 20930997 | C | A |
| 380 | CHR38 | 23702640 | G | C |
| 381 | CHRX | 4245861 | G | A |

As described above, by verifying the differences between individuals using 381 SNPs on the gene, individuals can be accurately and easily identified.

Although embodiments of the present disclosure have been described, the present disclosure is not limited to the embodiments disclosed herein and can be manufactured in various different forms. It will be understood by those skilled in the art that the disclosure may be embodied in other concrete forms without changing the technological scope and essential features. Therefore, the above-described embodiments should be considered only as examples in all aspects and not for purposes of limitation.

### Advantageous Effects

The present disclosure provides an SNP marker set for dog identification and a dog identification method using the same, and more specifically, can accurately and easily readably identify individual dogs through the SNP markers on the gene and combinations of the SNPs based on the simple results of AA/AB/BB (Homozygote/Heterozygote/Homo mutant) by using 381 single nucleotide polymorphisms (SNPs) on the gene, thereby proving to be useful for animal individual identification.

1. An SNP marker set for dog identification including all 381 SNPs listed in Table 41 in a dog genome dataset.

2. The SNP marker set for dog identification according to claim 1, wherein the breed of a dog is selected from a group including Beagle, Water dog, Maltese, Pomeranian, Poodle, Yorkshire Terrier, Shih Tzu, Dachshund, Chihuahua, American Pitbull Terrier, and crossbreeds thereof.

3. A composition for dog identification including an agent capable of detecting or amplifying the SNP marker set for dog identification of claim 1.

4. The composition for dog identification according to claim 3, wherein the agent is a primer which specifically binds to SNP markers or binds to amplify polynucleotides containing the SNP markers.

5. The composition for dog identification according to claim 3, wherein the agent is a probe which specifically binds to SNP markers or binds to amplify polynucleotides containing the SNP markers.

6. A kit for dog identification including the composition for dog identification of claim 3.

7. A microarray for dog identification including the composition for dog identification of claim 3.

8. A dog identification method comprising:
operation i) of extracting nucleic acids from a dog intended for individual identification; and
operation ii) of confirming the SNP marker set in the extracted nucleic acids.

9. The dog identification method according to claim 8, wherein the nucleic acids are extracted from one or more samples selected from a group consisting of the blood, epidermis, urine, feces, hair, saliva, oral cells, muscles, and organs of the dog.

10. The dog identification method according to claim 8, further comprising:
operation, after operation ii), of verifying one or more pieces of information selected from a group consisting of the blood type, physical characteristics, weight, sex, and age of the dog.
